(19) United States
(12) Patent Application Publication
(10) Pub. No.: US 2022/0354706 A1
(43) Pub. Date: Nov. 10, 2022

(54) Anatomically Contoured Dental Pad

(71) Applicant: Ramu Vuppala, Perrysburg, OH (US)

(72) Inventor: Ramu Vuppala, Perrysburg, OH (US)

(21) Appl. No.: 17/313,741

(22) Filed: May 6, 2021

(51) Int. Cl. A61F 13/20; A61F 13/45

(52) U.S. Cl. CPC A61F 13/2008; A61F 13/45; A61F 2013/53035

(57) ABSTRACT

An anatomically contoured dental pad constructed from an absorbent material configured to have an anatomically contoured profile. In some embodiments of the present invention the contour may have a profile or cross-section substantially similar to at least one of an hourglass and/or peanut shape, a capital “L” shape, or a capital “C” shape. Such a contoured profile beneficially aligns with the anatomical structures found in the mouth, such as the patient's tongue and/or gum line.

### CROSS-REFERENCES TO RELATED APPLICATIONS

Not Applicable.

### FIELD OF INVENTION

The present invention relates generally to an anatomically contoured dental pad for uses in dentistry. More particularly, the present invention is related to an absorbent pad designed to keep teeth and dental restoration materials (e.g. fillings) dry and isolated during and after dental restoration procedures.

### BACKGROUND OF THE INVENTION

Within the field of dentistry, cotton rolls are typically used. Commonly available cotton rolls have a basic cylindrical profile and have a relatively small diameter.

The use of conventional cotton rolls presents a significant risk of choking, due to their small diameter and their placement within the patient's mouth. The accumulation of saliva during a procedure may slicken conventional cotton rolls, causing them to become displaced and potentially fall through the pharynx, leading to choking and serious, even life-threatening complications. Further, conventional cotton rolls fail to conform to the contours of the oral cavity, further contributing to their tendency to become dislodged.

### SUMMARY OF THE INVENTION

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

Some embodiments of the present invention may be constructed from an absorbent material configured to have an anatomically contoured profile. In some embodiments of the present invention the contour may have a profile or cross-section substantial similar to an hourglass and/or peanut shape. In some other embodiments of the present invention the contour may have a profile or cross-section similar to the letter “L” or the letter “C”. Such contoured profiles beneficially align with the anatomical structures found in the mouth, such as the patient's tongue and/or gum line. The present invention better aids with isolation than regular cotton roll, as the present invention fits better due to its anatomically contoured shape. Additionally, they reduce the risk of choking as they are too big to swallow, thus preventing further injury to patients.

### BRIEF DESCRIPTION OF THE DRAWINGS

The present invention is described in detail below with reference to the attached drawing figures, wherein:

FIG. 1A illustrates a view of an embodiment of an anatomically contoured dental pad.

FIG. 1B illustrates a cross-sectional view of an embodiment of an anatomically contoured dental pad.

FIG. 1C illustrates an embodiment of an anatomically contoured dental pad placed in a patient's mouth.

FIG. 2A illustrates a view of an alternative embodiment of an anatomically contoured dental pad.

FIG. 2B illustrates a cross-sectional view of an alternative embodiment of an anatomically contoured dental pad.

FIG. 2C illustrates an alternative embodiment of an anatomically contoured dental pad placed in a patient's mouth.

FIG. 3A illustrates a view of a further embodiment of an anatomically contoured dental pad.

FIG. 3B illustrates a cross-sectional view of a further embodiment of an anatomically contoured dental pad.

FIG. 3C illustrates a further embodiment of an anatomically contoured dental pad placed in a patient's mouth.

### DETAILED DESCRIPTION OF THE INVENTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

The present invention is directed to an absorbent dental pad for use in dentistry. Some embodiments of the present invention may be constructed from an absorbent material configured to have an anatomically contoured profile. In some embodiments of the present invention the contour may have a profile or cross-section substantially similar to an hourglass and/or peanut shape. Such a contoured profile beneficially aligns with the anatomical structures found in the mouth, such as the patient's tongue and/or gum line.

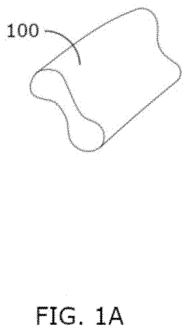

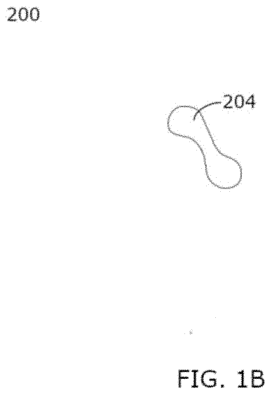

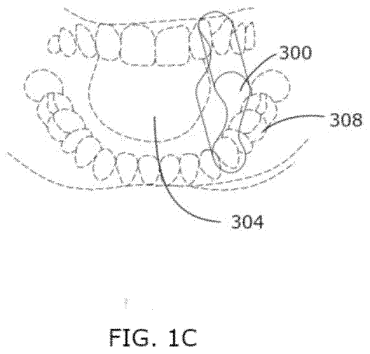

With reference to FIG. 1A, a view of an embodiment of an anatomically contoured dental pad 100 is given. The present invention may be constructed of any absorbent material which exhibits sufficient wicking action as to prevent the collection of moisture or saliva. One having skill in the art will recognize that suitable absorbent material may include, but are not limited to, cotton wool, quilted cotton, gauze, as well as acrylate polymers, such as sodium polyacrylate water-soluble thickeners and the superabsorbent polymer (SAP).

FIG. 1B provides a cross-sectional view 200 of an embodiment of an anatomically contoured dental pad. As depicted herein, the cross-sectional profile of dental pad 204 can be seen to be shaped substantially like an hourglass.

FIG. 10 provides an embodiment of an anatomically contoured dental pad placed in a patient's mouth. As depicted, dental pad 300 is positioned between the patient's tongue 304 and the patient's teeth 308. The contoured profile of dental pad 300 allow it to fit comfortably in the patient's mouth without becoming displaced, while the increased size of dental pad 300, relative to traditional cotton rolls, decreases the likelihood of choking. Embodiments of the present invention may be produced in various sizes as to better fit different size mouths.

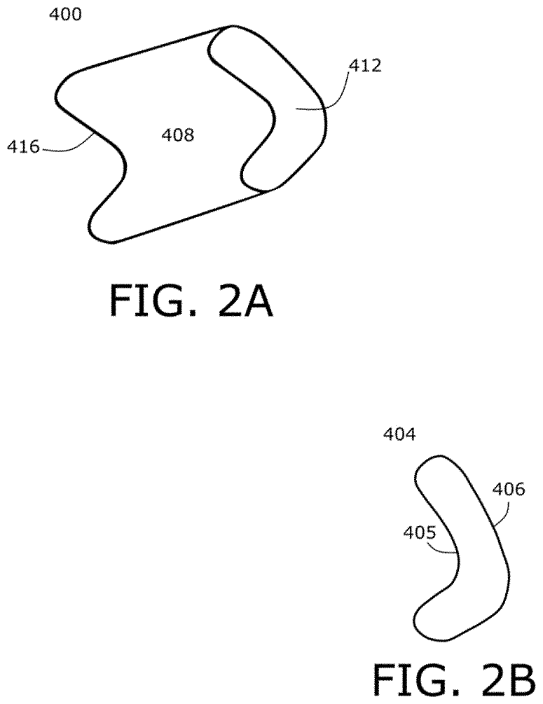

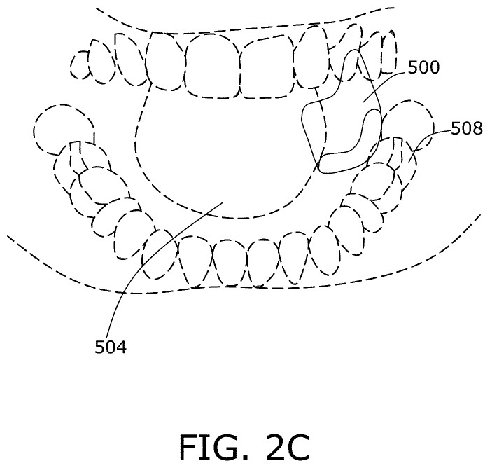

With reference to FIG. 2A, a view of an alternative embodiment of an anatomically contoured dental pad 400 is provided. FIG. 2B provides a cross-sectional view of the alternative embodiment of an anatomically contoured dental pad 404, having an interior side 405, and an exterior side 406. As depicted in FIG. 2B, the cross-sectional view of such embodiments may have a contour similar to a capital letter “L”. With continued reference to FIG. 2A, the interior side of the dental pad 408 may be constructed to include a further lengthwise concave contour along the length of the dental pad 404. As used herein, the term “lengthwise” refers to the dimension spanning from the front end of the pad 412 to the back end of the pad 416. Such an additional contour may provide for better fit of the pad, for example, along the patient's tongue, leading to additional comfort for the patient and a lower likelihood of the pad becoming displaced.

In some embodiments of the present invention, dental pad may include a stiffener to maintain its contour. Such stiffeners may include, but are not limited to, cellulose-based materials, such as paper or cardboard, or food-safe, semirigid plastics.

FIG. 2C illustrates the alternative embodiment of an anatomically contoured dental pad placed in a patient's mouth. As depicted, dental pad 500 is positioned between the patient's tongue 504 and the patient's teeth 508.

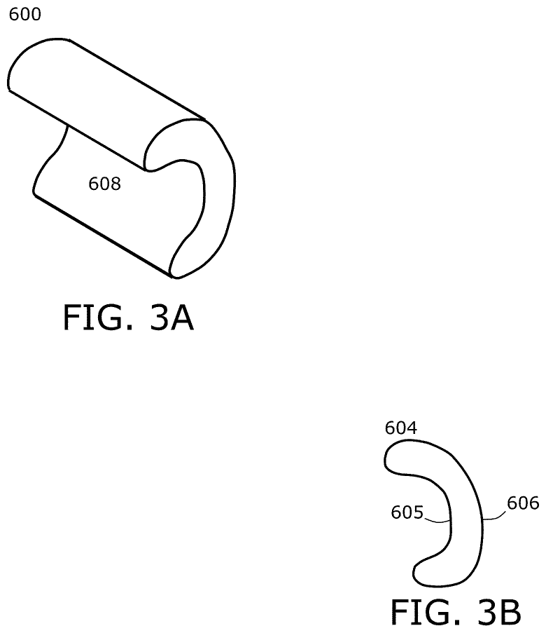

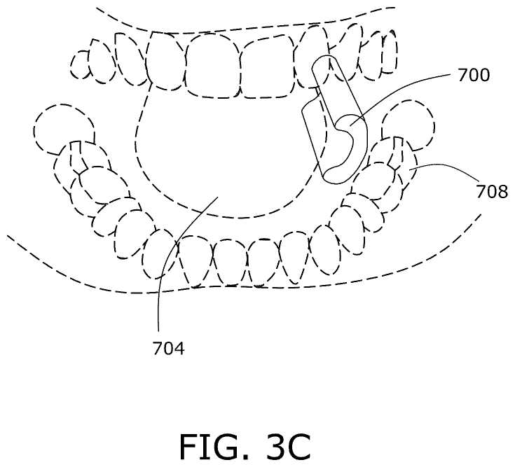

With reference to FIG. 3A, a view of an alternative embodiment of an anatomically contoured dental pad 600 is provided. FIG. 3B provides a cross-sectional view of the alternative embodiment of an anatomically contoured dental pad 604, having an interior side 605, and an exterior side 606. As depicted in FIG. 3B, the cross-sectional view of such embodiments may have a contour similar to a capital letter “C”. With continued reference to FIG. 3A, the interior side of the dental pad 608 may be constructed to include a further lengthwise concave contour along the length of the dental pad 604, similar to that discussed with reference to FIG. 2A.

FIG. 3C illustrates the alternative embodiment of an anatomically contoured dental pad placed in a patient's mouth. As depicted, dental pad 700 is positioned between the patient's tongue 704 and the patient's teeth 708.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

What is claimed is:

1. An anatomically contoured dental pad comprising:
a dental pad having a contoured cross-sectional profile,
the dental pad comprising an absorbent material.

2. The anatomically contoured dental pad of independent claim 1, wherein:
the absorbent material is cotton wool.

3. The anatomically contoured dental pad of independent claim 1, wherein:
the absorbent material is quilted cotton.

4. The anatomically contoured dental pad of independent claim 1, wherein:
the absorbent material is gauze.

5. The anatomically contoured dental pad of independent claim 1, wherein:
the absorbent material is an acrylate polymer.

6. The anatomically contoured dental pad of independent claim 1, wherein:
the contoured cross-sectional profile in substantially hourglass shaped.

7. The anatomically contoured dental pad of independent claim 1, wherein:
the contoured cross-sectional profile in substantially similar to a capital letter “C”.

8. The anatomically contoured dental pad of independent claim 1, wherein:
the contoured cross-sectional profile in substantially similar to a capital letter “L”.

9. The anatomically contoured dental pad of independent claim 1, further comprising:
a stiffener.

10. The anatomically contoured dental pad of claim 9, wherein:
the stiffener comprises a cellulose-based material.

11. The anatomically contoured dental pad of claim 10, wherein:
the cellulose-base material is at least one of a cardboard and a paper.

12. The anatomically contoured dental pad of claim 9, wherein:
the stiffener comprises a food-safe semirigid plastic.
